(19) United States
(12) Patent Application Publication
(10) Pub. No.: US 2021/0311065 A1
(43) Pub. Date: Oct. 7, 2021

(54) LIPID VESICLE-COATED MAGNETIC BEADS AND USES OF SAME

(71) Applicant: CELLMAX LIFE, INC., Sunnyvale, CA (US)

(72) Inventors: Rui Mei, Santa Clara, CA (US); Jen-Chia Wu, Taipei (TW)

(21) Appl. No.: 17/265,655

(22) PCT Filed: Aug. 2, 2019
(86) PCT No.: PCT/US2019/045005

§ 371 Date: Feb. 3, 2021

Related U.S. Application Data
(63) Continuation of application No. 16/054,091, filed on Aug. 3, 2018, now abandoned.

(51) Int. Cl. G01N 33/574; C12Q 1/6886; G01N 33/543

(52) U.S. Cl. CPC G01N 33/57492; C12Q 2600/112; G01N 33/5434; C12Q 1/6886

(57) ABSTRACT

Provided herein are lipid vesicle-coated magnetic beads, and methods of making and using the same.

### CROSS-REFERENCE TO RELATED APPLICATIONS

This application claims priority to U.S. patent application Ser. No. 16/054,091, filed Aug. 3, 2018; the entire contents of which are herein incorporated by reference.

### TECHNICAL FIELD

The present disclosure relates to the fields of molecular biology and the purification of target cells.

### BACKGROUND

Over 90% of all cancer-related deaths are caused by metastasis, the multi-step process by which cancer cells migrate from one site to another, often distant, site. During metastasis, cancer cells gain enhanced motility and enter the lymphatic system and bloodstream. Recent studies have shown that circulating tumor cells can be detected in blood samples. While efforts to detect even smaller numbers of circulating tumor cells have increased, it remains challenging to capture and isolate viable circulating tumor cells.

### SUMMARY

Without wishing to be bound by theory, the present invention is based on the discovery that the lipid-coated magnetic beads provided herein can provide for the efficient capture of desired target cells (e.g., cancer cells or any of the other types of target cells described herein), e.g., while maintaining the viability of the target cells.

Provided herein are compositions that include: (i) a magnetic bead having attached to its exterior surface a plurality of first binding partners; (ii) a plurality of lipid vesicles that comprise a plurality of the first binding partners on its exterior surface; (iii) a plurality of second binding partners; and (iv) a plurality of agents that bind specifically to a target cell, wherein each agent comprises an attached first binding partner; where: each of the plurality of second binding partners is capable of specifically binding to one or more first binding partners, a first subset of the plurality of the second binding partners specifically binds to (i) a first binding partner attached to the exterior surface of the magnetic bead and (ii) a first binding partner on the exterior surface of a lipid vesicle; a second subset of the plurality of the second binding partners specifically binds to (i) a first binding partner on the exterior surface of a lipid vesicle, and (ii) a first binding partner attached to an agent that binds specifically to a target cell. In some embodiments of any of the compositions provided herein, the lipid vesicles are non-fouling lipid vesicles. In some embodiments of any of the compositions provided herein, the non-fouling lipid vesicles include a zwitterionic lipid molecule. In some embodiments of any of the compositions provided herein, the non-fouling lipid vesicles include polyelectrolyte multilayers (PEMs) or a polymer brush. In some embodiments of any of the compositions provided herein, the PEMs include one or more of: poly-L-lysine, poly-L-glutamic acid, and poly-L-aspartic acid. In some embodiments of any of the compositions provided herein, the polymer brush includes [2-acryloyloxy)ethyl] trimethyl ammonium chloride (TMA) and 2-carboxyethyl acrylate (CAA). In some embodiments of any of the compositions provided herein, the vesicles include 1-palmitoyl-2-oleoyl-sn-glycero-3-phosphocholine (POPC) and 1,2-dipalmitoyl-sn-glycero-3-phosphoethanolamine-N-cap-biotinyl (b-PE), and biotin is the first binding partner. In some embodiments of any of the compositions provided herein, the lipid vesicles include POPC and b-PE at a ratio of 85:15. The chemical structures of 1-palmitoyl-2-oleoyl-sn-glycero-3-phosphocholine (POPC) and 1,2-dipalmitoyl-sn-glycero-3-phosphoethanolamine-N-cap-biotinyl (b-PE) are shown in FIG. 5.

In some embodiments of any of the compositions provided herein, the magnetic bead has covalently attached to its exterior surface the plurality of first binding partners. In some embodiments of any of the compositions provided herein, the magnetic bead has non-covalently attached to its exterior surface the plurality of first binding partners. In some embodiments of any of the compositions provided herein, the plurality of agents that bind specifically to a target cell each include a covalently attached first binding partner. In some embodiments of any of the compositions provided herein, the plurality of agents that bind specifically to a target cell each include a non-covalently attached first binding partner. In some embodiments of any of the compositions provided herein, the first binding partner includes biotin or a derivative thereof. In some embodiments of any of the compositions provided herein, the second binding partner includes avidin or a derivative thereof.

In some embodiments of any of the compositions provided herein, the plurality of agents that bind specifically to the target cell is an antibody or an antigen-binding fragment thereof. In some embodiments of any of the compositions provided herein, the target cell is a cancer cell, and the plurality of agents that bind specifically to the target cell is an antibody or an antigen-binding fragment thereof that specifically binds to a cancer antigen. In some embodiments of any of the compositions provided herein, the cancer antigen is epithelial cell adhesion molecule (EpCAM). In some embodiments of any of the compositions provided herein, the first binding partner binds to the second binding partner with a disassociation constant (K_{D}) of ≤10⁻⁷ M. In some embodiments of any of the compositions provided herein, the first binding partner binds to the second binding partner with disassociation constant (K_{D}) of ≤10⁻⁹ M.

Also provided herein are kits that include any of the compositions provided herein.

Also provided herein are methods of isolating a target cell from a biological sample that include: (a) contacting a biological sample including a target cell and non-target cells with any of the compositions provided herein; (b) after (a), washing the magnetic bead with a wash buffer under conditions sufficient to allow the association between (i) the first binding partner and the second binding partner to form a complex, and (ii) the agent that binds specifically to the target cell and the complex; and (c) after (b), applying a magnetic force to the magnetic bead under conditions sufficient to allow the association between (i) the first binding partner and the second binding partner, and (ii) the target cell and the agent that binds specifically to the target cell, thereby isolating the target cell. In some embodiments of any of the methods provided herein, the isolated target cell is viable. In some embodiments of any of the methods provided herein, the target cell is a circulating tumor cell or a circulating tumor stem cell. Some embodiments of any of the methods provided herein further include: (d) contacting the magnetic bead with an elution buffer under conditions that allow for the disassociation between the target cell and the agent that binds specifically to the target cell, thereby releasing the target cell from the magnetic bead.

In some embodiments of any of the methods provided herein, the biological sample includes blood. In some embodiments of any of the methods provided herein, the biological sample was obtained from a subject that has been diagnosed as having a cancer. In some embodiments of any of the methods provided herein, the biological sample was obtained from a subject that is suspected of having a cancer. In some embodiments of any of the methods provided herein, the wash buffer includes phosphate buffered saline and bovine serum albumin. In some embodiments of any of the methods provided herein, the wash buffer includes 1% w/v bovine serum albumin.

Some embodiments of any of the methods provided herein further include: (d) extracting a nucleic acid from the enriched target cell in step (c). Some embodiments of any of the methods provided herein further include: (e) genotyping the nucleic acid extracted from the enriched target cell in step (d). Some embodiments of any of the methods provided herein further include: (f) selecting or administering a pharmaceutical treatment to a subject based specifically on the genotype of the nucleic acid extracted from the enriched target cell in step (e). In some embodiments of any of the methods provided herein, the enriched isolated target cell is viable.

Also provided herein are methods of generating a magnetic bead having attached to its exterior surface a plurality of vesicles that include: (a) applying a magnetic field to a composition including: (i) a magnetic bead having attached to its exterior surface a plurality of first binding partners; (ii) a plurality of lipid vesicles that comprise a plurality of the first binding partners on its exterior surface; (iii) a plurality of second binding partners; and (iv) a plurality of agents that bind specifically to a target cell, where each agent includes an attached first binding partner; where: each of the plurality of second binding partners is capable of specifically binding to one or more first binding partners, a first subset of the plurality of the second binding partners specifically binds to (i) a first binding partner attached to the exterior surface of the magnetic bead and (ii) a first binding partner on the exterior surface of a lipid vesicle; a second subset of the plurality of the second binding partners specifically binds to (i) a first binding partner on the exterior surface of a lipid vesicle, and (ii) a first binding partner attached to an agent that binds specifically to a target cell, where the magnetic field is applied under conditions sufficient to allow the association between (i) the first binding partner and the second binding partner; (b) after step (a), washing the magnetic bead with a wash buffer under conditions sufficient to allow the association between the first binding partner and the second binding partner; and (c) after step (b), resuspending the washed beads with an aqueous solution comprising between 1% and 10% bovine serum albumin under conditions that allow the association between the first binding partner and the second binding partner, thereby generating a magnetic bead having attached to its exterior surface a plurality of lipid vesicles.

Also provided herein are methods of generating a magnetic bead having attached to its exterior surface a plurality of lipid vesicles that include: (a) incubating: (i) a magnetic bead having attached to its exterior surface a plurality of first binding partners; (ii) a plurality of lipid vesicles that comprise a plurality of the first binding partners on its exterior surface; and (iii) a plurality of second binding partners; where: each of the plurality of second binding partners is capable of specifically binding to one or more first binding partners, a subset of the plurality of the second binding partners specifically binds to (i) a first binding partner attached to the exterior surface of the magnetic bead and (ii) a first binding partner on the exterior surface of a lipid vesicle; under conditions sufficient to allow the association between (i) the first binding partner and the second binding partner; (b) after step (a), washing the magnetic bead with a wash buffer under conditions sufficient to allow the association between the first binding partner and the second binding partner; (c) after (b), contacting the magnetic bead with a plurality of agents that bind specifically to a target cell, wherein each agent includes an attached first binding partner, under conditions sufficient to allow the association between the first binding partner and the second binding partner, thereby generating a magnetic bead having attached to its exterior surface a plurality of lipid vesicles.

In some embodiments of any of the methods provided herein, the lipid vesicles are non-fouling lipid vesicles. In some embodiments of any of the methods provided herein, the lipid vesicles include a zwitterionic lipid molecule. In some embodiments of any of the methods provided herein, the lipid vesicles comprise polyelectrolyte multilayers (PEMs) or a polymer brush. In some embodiments of any of the methods provided herein, the PEMs include one or more of: poly-L-lysine, poly-L-glutamic acid, and poly-L-aspartic acid. In some embodiments of any of the methods provided herein, the polymer brush includes [2-acryloyloxy)ethyl] trimethyl ammonium chloride (TMA) and 2-carboxyethyl acrylate (CAA). In some embodiments of any of the methods provided herein, the lipid vesicles include 1-palmitoyl-2-oleoyl-sn-glycero-3-phosphocholine (POPC) and 1,2-dipalmitoyl-sn-glycero-3-phosphoethanolamine-N-cap-biotinyl (b-PE), and biotin is the first binding partner. In some embodiments of any of the methods provided herein, the lipid vesicles include POPC and b-PE at a molar ratio of 85:15. In some embodiments of any of the methods provided herein, the magnetic bead has attached to its exterior surface the plurality of first binding partners. In some embodiments of any of the methods provided herein, the magnetic bead has non-covalently attached to its exterior surface the plurality of first binding partners. In some embodiments of any of the methods provided herein, the plurality of agents that bind specifically to a target cell each include a covalently attached first binding partner. In some embodiments of any of the methods provided herein, the plurality of agents that bind specifically to a target cell each include a non-covalently attached first binding partner. In some embodiments of any of the methods provided herein, the first binding partner includes biotin or a derivative thereof. In some embodiments of any of the methods provided herein, the second binding partner includes avidin or a derivative thereof.

In some embodiments of any of the methods provided herein, the plurality of agents that bind specifically to the target cell is an antibody or an antigen-binding fragment thereof. In some embodiments of any of the methods provided herein, the target cell is a cancer cell, and the plurality of agents that bind specifically to the target cell is an antibody or an antigen-binding fragment thereof that specifically binds to a cancer antigen. In some embodiments of any of the methods provided herein, the cancer antigen is epithelial cell adhesion molecule (EpCAM). In some embodiments of any of the methods provided herein, the first binding partner binds to the second binding partner with a disassociation constant (K_{D}) of ≤10⁻⁷ M. In some embodiments of any of the methods provided herein, the first binding partner binds to the second binding partner with disassociation constant (K_{D}) of ≤10⁻⁹ M. In some embodiments of any of the methods provided herein, the wash buffer includes phosphate buffered saline and bovine serum albumin. In some embodiments of any of the methods provided herein, the wash buffer includes 1% w/v bovine serum albumin.

Also provided herein is a magnetic bead having attached to its exterior surface a plurality of lipid vesicles produced by any of the methods provided herein.

As used herein, the term “non-fouling lipid vesicle” means a lipid vesicle that includes a non-fouling lipid. In some embodiments, a non-fouling lipid vesicle can include 1-palmitoyl-2-oleoyl-sn-glycero-3-phosphocholine (POPC) lipid.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

FIG. 1 is a schematic representation of an exemplary embodiment of the compositions and methods described herein.

FIG. 2 is a schematic representation of an exemplary embodiment of the methods provided herein.

FIG. 3 is a graph showing the overall recovery (%) of an exemplary target cell from a sample comprising white blood cells (WBCs).

FIG. 4 is a table comparing the recovery rate of target cells and the purity of target cells using (1) a conventional 2D chip method of isolating a target cell or (2) an exemplary embodiment of the methods provided herein (e.g., high throughput 3D platform).

FIG. 5 is the chemical structures of 1-palmitoyl-2-oleoyl-sn-glycero-3-phosphocholine (POPC) and 1,2-dipalmitoyl-sn-glycero-3-phosphoethanolamine-N-cap-biotinyl (b-PE).

### DETAILED DESCRIPTION

The compositions, kits, and methods described herein can be used to capture, isolate, or enrich specific and/or rare populations of target cells (e.g., any of the exemplary target cells described herein) from a biological sample (e.g., a biological sample comprising blood, serum, or plasma), which optionally, can thereafter be cultured and/or used for further analysis (e.g., genotyping or DNA sequencing).

Provided herein are compositions that include: (i) a magnetic bead (e.g., any of the exemplary magnetic beads described herein or known in the art) having attached to its exterior surface a plurality of first binding partners (e.g., any of the exemplary first binding partners described herein); (ii) a plurality of non-fouling lipid vesicles (e.g., any of the exemplary lipid vesicles described herein or known in the art) that comprise a plurality of the first binding partners on its exterior surface; (iii) a plurality of second binding partners (e.g., any of the exemplary second binding partners described herein or known in the art); and (iv) a plurality of agents that bind specifically to a target cell, wherein each agent comprises an attached first binding partner (e.g., any of the exemplary agents that bind specifically to a target cell described herein); wherein: each of the plurality of second binding partners is capable of specifically binding to at least two different first binding partners; a first subset of the plurality of the second binding partners specifically binds to (i) a first binding partner covalently attached to the exterior surface of the magnetic bead and (ii) a first binding partner on the exterior surface of a non-fouling lipid vesicle; and a second subset of the plurality of the second binding partners specifically binds to (i) a first binding partner on the exterior surface of a non-fouling lipid vesicle, and (ii) a first binding partner covalently attached to an agent that binds specifically to a target cell.

Also provided herein are methods of isolating a target cell from a biological sample that include: (a) contacting a biological sample (e.g., any of the exemplary biological samples described herein or known in the art) comprising a target cell (e.g., any of the exemplary target cells described herein or known in the art) and non-target cells with any of the compositions provided herein; (b) after (a), washing the magnetic bead with a wash buffer under conditions sufficient to allow the association between (i) the first binding partner and the second binding partner, and (ii) the target cell and the agent that binds specifically to the target cell, and sufficient to substantially not allow for the association between the non-target cells and the agent that binds specifically to the target cell; and (c) after (b), applying a magnetic force to isolate the magnetic bead under conditions sufficient to allow the association between (i) the first binding partner and the second binding partner, and (ii) the target cell and the agent that binds specifically to the target cell, thereby isolating the target cell.

The methods provide for isolation of a cell population that is greater than 90%, greater than 92%, greater than 94%, greater than 95%, greater than 96%, greater than 96.5%, greater than 97%, greater than 97.5%, greater than 98%, greater than 98.5%, greater than 99%, greater than 99.1%, greater than 99.2%, greater than 99.3%, greater than 99.4%, greater than 99.5%, greater than 99.6%, greater than 99.7%, greater than 99.8%, greater than 99.9%, or 100% of the target cells (e.g., viable target cells).

The methods provide for the isolation of a target cell population from a biological sample, where the target cells are at least 90%, at least 91%, at least 92%, at least 93%, at least 94%, at least 95%, at least 96%, at least 97%, at least 98%, at least 99%, or 100% viable.

Also provided herein are methods of generating a magnetic bead (e.g., any of the exemplary magnetic beads described herein or known in the art) having attached to its exterior surface a plurality of non-fouling lipid vesicles (e.g., any of the exemplary non-fouling lipid vesicles described herein or known in the art) that include: (a) applying a magnetic field to a composition including: (i) a magnetic bead having attached to its exterior surface a plurality of first binding partners (e.g., any of the first binding partners described herein); (ii) a plurality of non-fouling lipid vesicles that comprise a plurality of the first binding partners on its exterior surface; (iii) a plurality of second binding partners (e.g., any of the exemplary second binding partners described herein or known in the art); and (iv) a plurality of agents (e.g., any of the exemplary agent described herein) that bind specifically to a target cell (e.g., any of the exemplary target cells described herein or known in the art), wherein each agent includes an attached first binding partner; where: each of the plurality of second binding partners is capable of specifically binding to one or more (e.g., two or more) different first binding partners, a first subset of the plurality of the second binding partners specifically binds to (i) a first binding partner covalently or non-covalently attached to the exterior surface of the magnetic bead and (ii) a first binding partner on the exterior surface of a non-fouling lipid vesicle; a second subset of the plurality of the second binding partners specifically binds to (i) a first binding partner on the exterior surface of a non-fouling lipid vesicle, and (ii) a first binding partner attached to an agent that binds specifically to a target cell, where the magnetic field is applied under conditions sufficient to allow the association between (i) the first binding partner and the second binding partner; (b) after step (a), washing the magnetic bead with a wash buffer under conditions sufficient to allow the association between the first binding partner and the second binding partner; and (c) after step (b), resuspending the washed beads with an aqueous solution (e.g., an aqueous solution including between 1% and 10% bovine serum albumin) under conditions that allow the association between the first binding partner and the second binding partner, thereby generating a magnetic bead having attached to its exterior surface a plurality of non-fouling lipid vesicles.

Also provided herein are methods of generating a magnetic bead having attached to its exterior surface a plurality of non-fouling lipid vesicles that include: (a) incubating: (i) a magnetic bead (e.g., any of the exemplary magnetic beads described herein or known in the art) having attached to its exterior surface a plurality of first binding partners (e.g., any of the exemplary first binding partners described herein or known in the art); (ii) a plurality of non-fouling lipid vesicles (e.g., any of the exemplary non-fouling lipid vesicles described herein or known in the art) that include a plurality of the first binding partners on its exterior surface; and (iii) a plurality of second binding partners (e.g., any of the exemplary second binding partners described herein or known in the art); where: each of the plurality of second binding partners is capable of specifically binding to one or more (e.g., two or more) different first binding partners, a subset of the plurality of the second binding partners specifically binds to (i) a first binding partner covalently or non-covalently attached to the exterior surface of the magnetic bead and (ii) a first binding partner on the exterior surface of a non-fouling lipid vesicle; under conditions sufficient to allow the association between (i) the first binding partner and the second binding partner; (b) after step (a), washing the magnetic bead with a wash buffer under conditions sufficient to allow the association between the first binding partner and the second binding partner; (c) after (b), contacting the magnetic bead with a plurality of agents (e.g., any of the exemplary agents that bind specifically to a target cell described herein or known in the art) that bind specifically to a target cell (e.g., any of the exemplary target cells described herein or known in the art), where each agent includes an attached first binding partner, under conditions sufficient to allow the association between the first binding partner and the second binding partner, thereby generating a magnetic bead having attached to its exterior surface a plurality of non-fouling lipid vesicles.

Non-limiting aspects of these methods are described below, and can be used in any combination without limitation. Additional aspects of these methods are known in the art.

### Compositions

Provided herein are compositions that include: (i) a magnetic bead (e.g., any of the exemplary magnetic beads described herein or known in the art) having attached to its exterior surface a plurality of first binding partners (e.g., any of the exemplary first binding partners described herein or known in the art); (ii) a plurality of lipid vesicles (e.g., any of the exemplary lipid vesicles described herein or known in the art) that include a plurality of the first binding partners on its exterior surface; (iii) a plurality of second binding partners (e.g., any of the exemplary second binding partners described herein or known in the art); and (iv) a plurality of agents (e.g., any of the exemplary agents described herein or known in the art) that bind specifically to a target cell (e.g., any of the exemplary target cells described herein or known in the art), where each agent includes an attached first binding partner; where: each of the plurality of second binding partners is capable of specifically binding to one or more (e.g., two or more) (e.g., one, two, three, four, or five) different first binding partners, a first subset of the plurality of the second binding partners specifically binds to (i) a first binding partner covalently or non-covalently attached to the exterior surface of the magnetic bead and (ii) a first binding partner on the exterior surface of a lipid vesicle; a second subset of the plurality of the second binding partners specifically binds to (i) a first binding partner on the exterior surface of a non-fouling lipid vesicle, and (ii) a first binding partner covalently attached to an agent that binds specifically to a target cell.

In some embodiments of any of the compositions described herein, the lipid vesicles are non-fouling lipid vesicles. In some embodiments of any of the compositions described herein, the lipid vesicles include zwitterionic lipid molecules. In some embodiments of these compositions, the lipid vesicles comprise polyelectrolyte multilayers (PEMs) or a polymer brush. In some embodiments of these compositions, the PEMs comprise one or more of: poly-L-lysine, poly-L-glutamic acid, and poly-L-aspartic acid. In some embodiments of any of the compositions described herein, the polymer brush comprises [2-acryloyloxy)ethyl] trimethyl ammonium chloride (TMA) and 2-carboxyethyl acrylate (CAA). Additional aspects and examples of lipid vesicles are described herein. Non-limiting exemplary aspects of magnetic beads are also described herein.

Non-limiting examples and aspects of first and second binding partners and agents that bind specifically to a target cell are also described herein.

In some embodiments of any of the compositions described herein, the compositions can be disposed in a multi-well plate (e.g., a 96-well plate). In some embodiments, the compositions provided herein can be attached to a solid surface (e.g., a film, a chip, or a microfluidic channel).

In some embodiments of these compositions, the magnetic bead has covalently attached to its exterior surface, the plurality of first binding partners. In some embodiments of these compositions, the magnetic bead has non-covalently attached to its exterior surface, the plurality of first binding partners. In some embodiments of these compositions, the plurality of agents that bind specifically to a target cell each include a covalently attached first binding partner. In some embodiments of these compositions, the plurality of agents that bind specifically to a target cell each include a non-covalently attached first binding partner.

In some embodiments of these compositions, the lipid vesicles include 1-palmitoyl-2-oleoyl-sn-glycero-3-phosphocholine (POPC) and 1,2-dipalmitoyl-sn-glycero-3-phosphoethanolamine-N-cap-biotinyl (b-PE), and biotin is the first binding partner and avidin is the second binding partner. In some embodiments of these compositions, the first binding partner is avidin and the second binding partner is biotin.

### Magnetic Beads

In some embodiments of any of the compositions or methods described herein, the magnetic bead has a ferromagnetic core or a superparamagnetic core. In some examples, the magnetic bead has a core including a metal (e.g., Co, Fe, or Ni) or an oxide thereof. In some examples, the magnetic bead can include transition-metal-doped oxides and metal alloys, such as CoPt₃, FeCo, and FePt. In some examples, the magnetic bead can include an iron oxide such as magnetite (Fe₃O₄) or maghemite (γ-Fe₂O₃). The core of a magnetic bead can be formed using any of the methods described in U.S. Pat. Nos. 5,834,121, 5,395,688, 5,356,713, 5,318,797, 5,283,079, 5,232,7892, 5,091,206, 4,965,007, 4,774,265, 4,770,183, 4,654,267, 4,554,088, 4,490,436, 4,336,173, and 4,421,660.

In some examples, a magnetic bead can have a surface coating the core. In some examples, the magnetic bead can have a coating that includes one or more of alkanesulphonic acids, alkanephosphonic acids, oleic acids, lactobionic acid, lauric acid, alginate, chitosan, dextran, polyethylene glycol, polyvinyl alcohol, pullulan, and polyethylene imine. Additional materials that can be used to coat the core of a magnetic bead are known in the art. As used herein, “coat” can be a material that covers at least 90% of the outer surface of the core.

In some embodiments, the magnetic bead includes a polymer that coats the core of the magnetic bead. Non-limiting examples of polymers that can be used to coat the core of a magnetic bead include: polystyrenes, polyacrylamides, polyetherurethanes, polysulfones, fluoronated or chlorinated polymers such as polyvinyl chloride, polyethylenes and polypropylenes, polycarbonates and polyesters. Other polymers include polyolefins such as polybutadiene, polydichlorobutadiene, polyisoprene, polychloroprene, polyvinylidene halides, polyvinylidene carbonate, and polyfluorinated ethylenes. In some examples, a copolymer can be used to coat the core of a magnetic bead. Non-limiting examples of copolymers include styrene/butadiene, alpha-methyl styrene/dimethyl siloxane, and other polysiloxanes (e.g., polydimethyl siloxane, polyphenylmethyl siloxane, and polytrifluoropropylmethyl siloxane). In some examples, the core of the magnetic bead is coated with a polyacrylonitrile or an acrylonitrile-containing polymer (e.g., poly alpha-acrylanitrile copolymers, alkyd or terpenoid resins, and polyalkylene polysulfonates).

In some embodiments of any of the compositions or methods described herein, the magnetic bead has an average diameter of about 1 μm to about 140 μm (e.g., about 1 μm to about 120 μm, about 1 μm to about 100 μm, about 1 μm to about 80 μm, about 1 μm to about 60 μm, about 1 μm to about 40 μm, about 1 μm to about 20 μm, about 1 μm to about 10 μm, about 1 μm to about 5 μm, about 5 μm to about 140 μm, about 5 μm to about 120 μm, about 5 μm to about 100 μm, about 5 μm to about 50 μm, about 5 μm to about 25 μm, about 5 μm to about 15 μm, about 5 μm to about 10 μm, about 10 μm to about 140 μm, about 10 μm to about 120 μm, about 10 μm to about 100 μm, about 10 μm to about 50 μm, about 10 μm to about 25 μm, about 10 μm to about 20 μm, about 20 μm to about 140 μm, about 20 μm to about 120 μm, about 20 μm to about 100 μm, about 20 μm to about 50 μm, about 20 μm to about 40 μm, about 20 μm to about 30 μm, about 50 μm to about 140 μm, about 50 μm to about 120 μm, about 50 μm to about 100 μm, about 50 μm to about 75 μm, about 60 μm to about 140 μm, about 60 μm to about 120 μm, about 60 μm to about 100 μm, about 1 μm, about 5 μm, about 10 μm, about 15 μm, about 20 μm, about 30 μm, about 40 μm, about 60 μm, about 80 μm, about 100 μm, about 120 μm, or about 140 μm).

In some examples, the magnetic bead can have a spherical shape or an ellipsoidal shape. In some examples, the magnetic bead can have an icosahedron shape, a dodecahedron shape, an octahedron shape, a half-sphere shape, a cuboid shape, a hexagonal prism shape, or a torus shape. In some examples, the exterior surface of the magnetic bead can be smooth. In some examples, the exterior surface of the magnetic bead has one or more grooves. In some embodiments, the exterior surface of the magnetic bead is stipled.

In some embodiments, a population of magnetic beads in any of the compositions or methods described herein can be a mixture of magnetic beads having different properties (e.g., different diameters, different shapes, and/or different composition). In some embodiments, a population of magnetic beads in any of the compositions or methods described herein is a homogenous population of magnetic beads having substantially the same properties (e.g., approximately the same diameter, the same shape, and/or the same composition).

Various magnetic beads are commercially available and known in the art, and can be used in any of the compositions and/or methods described herein. The magnetic beads have attached to its exterior surface (e.g., by covalent or non-covalent attachments) a plurality of the first binding partners (e.g., any of the first binding partners described herein). In some embodiments, where the first binding partner is covalently attached to the exterior surface of the magnetic bead, the first binding partner and the surface of the magnetic bead are connected via a disulfide bond, an amide bond, an ester bond, an ether bond, a thioester bond, a phosphate ester bond, a phosphodiester bond, a hemiacetal bond, and a glycosidic bond. In some embodiments, the first binding partner is attached to the exterior surface of the magnetic bead via a non-covalent bond (e.g., an ionic or a hydrogen bond).

### Lipid Vesicles

The term “lipid vesicles” is understood to mean a structure comprising one or more lipid layers that encloses a volume of fluid. For example, a lipid vesicle can include a single lipid layer (a monolayer) that encloses a volume of fluid. In other examples, a lipid vesicle can include one or more lipid bilayers, where each bilayer includes two monolayers that each contain amphipathic lipid molecules oppositely oriented. Amphipathic lipids include a polar (hydrophilic) headgroup region covalently linked to one or two non-polar (hydrophobic) acyl chains. Energetically unfavorable contacts between the hydrophobic acyl chains and the surrounding aqueous medium induce the amphipathic lipid molecules to arrange themselves such that their polar headgroups are oriented towards the bilayer's surface, while the acyl chains reorient towards the interior of the bilayer. An energetically stable structure is thus formed in which the acyl chains are effectively shielded from coming into contact with the aqueous environment.

In some examples, the lipid vesicles have a single bilayer membrane (e.g., small unilamellar vesicles (SUVs) or large unilamellar vesicles (LUVs)). In some examples, the lipid vesicles have multiple bilayer membranes (e.g., multilamellar large vesicles (MLVs)).

As used herein the term “plurality of lipid vesicles” refers to at least 1×10¹ lipid vesicles, at least 1×10², at least 1×10³, at least 1×10⁴, at least 1×10⁵, at least 1×10⁶, at least 1×10⁷, at least 1×10⁸, or at least 1×10⁹ lipid vesicles. For example, any of the compositions described herein can include a plurality of, e.g., about 1×10¹ to about 1×10⁹ lipid vesicles, about 1×10¹ to about 1×10⁸ lipid vesicles, about 1×10¹ to about 1×10⁷ lipid vesicles, about 1×10¹ to about 1×10⁶ lipid vesicles, about 1×10¹ to about 1×10⁵ lipid vesicles, about 1×10¹ to about 1×10⁴ lipid vesicles, about 1×10¹ to about 1×10³ lipid vesicles, about 1×10² to about 1×10⁹ lipid vesicles, about 1×10² to about 1×10⁶ lipid vesicles, or about 1×10³ to about 1×10⁶ lipid vesicles.

In some embodiments of any of the compositions described herein, the lipid vesicles are non-fouling lipid vesicles.

In any of the embodiments described herein, the lipid vesicles can have an average diameter of about 10 nm to about 15 μm, about 10 nm to about 10 μm, about 10 nm to about 5 μm, about 10 nm to about 1 μm, about 10 nm to about 900 nm, about 10 nm to about 800 nm, about 10 nm to about 700 nm, about 10 nm to about 600 nm, about 10 nm to about 500 nm, about 10 nm to about 450 nm, about 10 nm to about 400 nm, about 10 nm to about 350 nm, about 10 nm to about 300 nm, about 10 nm to about 250 nm, about 10 nm to about 200 nm, about 10 nm to about 150 nm, about 10 nm to about 100 nm, about 10 nm to about 50 nm, about 10 nm to about 25 nm, about 25 nm to about 15 μm, about 25 nm to about 10 μm, about 25 nm to about 5 μm, about 25 nm to about 1 μm, about 25 nm to about 900 nm, about 25 nm to about 800 nm, about 25 nm to about 700 nm, about 25 nm to about 600 nm, about 25 nm to about 500 nm, about 25 nm to about 450 nm, about 25 nm to about 400 nm, about 25 nm to about 350 nm, about 25 nm to about 300 nm, about 25 nm to about 250 nm, about 25 nm to about 200 nm, about 25 nm to about 150 nm, about 25 nm to about 100 nm, about 25 nm to about 50 nm, about 50 nm to about 15 μm, about 50 nm to about 10 μm, about 50 nm to about 5 μm, about 50 nm to about 1 μm, about 50 nm to about 900 nm, about 50 nm to about 800 nm, about 50 nm to about 700 nm, about 50 nm to about 600 nm, about 50 nm to about 500 nm, about 50 nm to about 450 nm, about 50 nm to about 400 nm, about 50 nm to about 350 nm, about 50 nm to about 300 nm, about 50 nm to about 250 nm, about 50 nm to about 200 nm, about 50 nm to about 150 nm, about 50 nm to about 100 nm, about 100 nm to about 15 μm, about 100 nm to about 10 μm, about 100 nm to about 5 μm, about 100 nm to about 1 μm, about 100 nm to about 900 nm, about 100 nm to about 800 nm, about 100 nm to about 700 nm, about 100 nm to about 600 nm, about 100 nm to about 500 nm, about 100 nm to about 450 nm, about 100 nm to about 400 nm, about 100 nm to about 350 nm, about 100 nm to about 300 nm, about 100 nm to about 250 nm, about 100 nm to about 200 nm, about 100 nm to about 150 nm, about 150 nm to about 15 μm, about 150 nm to about 10 μm, about 150 nm to about 5 μm, about 150 nm to about 1 μm, about 150 nm to about 900 nm, about 150 nm to about 800 nm, about 150 nm to about 700 nm, about 150 nm to about 600 nm, about 150 nm to about 500 nm, about 150 nm to about 450 nm, about 150 nm to about 400 nm, about 150 nm to about 350 nm, about 150 nm to about 300 nm, about 150 nm to about 250 nm, about 150 nm to about 200 nm, about 200 nm to about 15 μm, about 200 nm to about 10 μm, about 200 nm to about 5 μm, about 200 nm to about 1 μm, about 200 nm to about 900 nm, about 200 nm to about 800 nm, about 200 nm to about 700 nm, about 200 nm to about 600 nm, about 200 nm to about 500 nm, about 200 nm to about 450 nm, about 200 nm to about 400 nm, about 200 nm to about 350 nm, about 200 nm to about 300 nm, about 200 nm to about 250 nm, about 250 nm to about 15 μm, about 250 nm to about 10 μm, about 250 nm to about 5 μm, about 250 nm to about 1 μm, about 250 nm to about 900 nm, about 250 nm to about 800 nm, about 250 nm to about 700 nm, about 250 nm to about 600 nm, about 250 nm to about 500 nm, about 250 nm to about 450 nm, about 250 nm to about 400 nm, about 250 nm to about 350 nm, about 250 nm to about 300 nm, about 300 nm to about 15 μm, about 300 nm to about 10 μm, about 300 nm to about 5 μm, about 300 nm to about 1 μm, about 300 nm to about 900 nm, about 300 nm to about 800 nm, about 300 nm to about 700 nm, about 300 nm to about 600 nm, about 300 nm to about 500 nm, about 300 nm to about 450 nm, about 300 nm to about 400 nm, about 300 nm to about 350 nm, about 350 nm to about 15 μm, about 350 nm to about 10 μm, about 350 nm to about 5 μm, about 350 nm to about 1 μm, about 350 nm to about 900 nm, about 350 nm to about 800 nm, about 350 nm to about 700 nm, about 350 nm to about 600 nm, about 350 nm to about 500 nm, about 350 nm to about 450 nm, about 350 nm to about 400 nm, about 400 nm to about 15 μm, about 400 nm to about 10 μm, about 400 nm to about 5 μm, about 400 nm to about 1 μm, about 400 nm to about 900 nm, about 400 nm to about 800 nm, about 400 nm to about 700 nm, about 400 nm to about 600 nm, about 400 nm to about 500 nm, about 400 nm to about 450 nm, about 450 nm to about 15 μm, about 450 nm to about 10 μm, about 450 nm to about 5 μm, about 450 nm to about 1 μm, about 450 nm to about 900 nm, about 450 nm to about 800 nm, about 450 nm to about 700 nm, about 450 nm to about 600 nm, about 450 nm to about 500 nm, about 500 nm to about 15 μm, about 500 nm to about 10 μm, about 500 nm to about 5 μm, about 500 nm to about 1 μm, about 500 nm to about 900 nm, about 500 nm to about 800 nm, about 500 nm to about 700 nm, about 500 nm to about 600 nm, about 600 nm to about 15 μm, about 600 nm to about 10 μm, about 600 nm to about 5 μm, about 600 nm to about 1 μm, about 600 nm to about 900 nm, about 600 nm to about 800 nm, about 600 nm to about 700 nm, about 700 nm to about 15 μm, about 700 nm to about 10 μm, about 700 nm to about 5 μm, about 700 nm to about 1 μm, about 700 nm to about 900 nm, about 700 nm to about 800 nm, about 800 nm to about 15 μm, about 800 nm to about 10 μm, about 800 nm to about 5 μm, about 800 nm to about 1 μm, about 800 nm to about 900 nm, about 900 nm to about 15 μm, about 900 nm to about 10 μm, about 900 nm to about 5 μm, about 900 nm to about 1 μm, about 1 μm to about 15 μm, about 1 μm to about 10 μm, about 1 μm to about 5 μm, about 5 μm to about 15 μm, about 5 μm to about 10 μm, or about 10 μm to about 15 μm.

In some embodiments, the lipid vesicles include phospholipids such as, e.g., 1-palmitoyl-2-oleoyl-glycero-3-phosphocholine (POPC), phosphatidylcholine (PC), phosphatidylserine (PS), phosphatidylethanolamine (PE), 1,2-dipalmitoylsn-glycero-3-phosphoethanolamine-N-cap-biotinyl (b-PE), phosphatidylglycerol (PG), phosphatidylinositol (PI), and phosphatidic acid (PA). In some embodiments, the lipid vesicles include, e.g., POPC and b-PE. In some embodiments, the lipid vesicles include, e.g., cholesterol and cholesterol-PEG. In some embodiments, the lipid vesicles can include, e.g., proteins, carbohydrates, or polyethyleneglycol (PEG).

In some embodiments, the lipid vesicles include one or more lipids. The type, number, and ratio of lipids can be varied as long as they are capable of forming the lipid vesicles. The lipids may be isolated from a naturally occurring source or they may be synthesized apart from any naturally-occurring source.

In some embodiments, at least one (or some) of the lipids is/are amphipathic lipids, defined as having a hydrophilic and a hydrophobic portion (typically a hydrophilic head and a hydrophobic tail). The hydrophilic portion may include polar or charged groups, such as carbohydrates, phosphate, carboxylic, sulfato, amino, sulfhydryl, nitro, hydroxyl, and other like groups. The hydrophobic portion may include apolar groups that include without limitation long chain saturated and unsaturated aliphatic hydrocarbon groups and groups substituted by one or more aromatic, cyclo-aliphatic, or heterocyclic group(s). Examples of amphipathic lipids include, but are not limited to, phospholipids, aminolipids, and sphingolipids.

In some examples, the lipid vesicles includes phospholipids. Phospholipids include without limitation phosphatidylcholine, phosphatidylethanolamine, phosphatidylglycerol, phosphatidylinositol, phosphatidylserine, and the like. It is to be understood that other lipid membrane components, such as cholesterol, sphingomyelin, cardiolipin, etc. can be included in the lipid vesicles.

The lipids present in a lipid vesicle can be anionic and neutral (including zwitterionic and polar) lipids including anionic and neutral phospholipids. Neutral lipids exist in an uncharged or neutral zwitterionic to form at a selected pH. At physiological pH, such lipids include, for example, dioleoylphosphatidylglycerol (DOPG), diacylphosphatidylcholine, diacylphosphatidylethanolamine, ceramide, sphingomyelin, cephalin, cholesterol, cerebrosides, and diacylglycerols. Examples of zwitterionic lipids include without limitation dioleoylphosphatidylcholine (DOPC), dimyristoylphosphatidylcholine (DMPC), and dioleoylphosphatidylserine (DOPS). An anionic lipid is a lipid that is negatively charged at physiological pH. These lipids include without limitation phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids.

In some examples, the lipid vesicles include anionic and neutral lipids (also called non-cationic lipids). Such lipids may contain phosphorus but they are not so limited. Examples of non-cationic lipids include lecithin, lysolecithin, phosphatidylethanolamine, lysophosphatidylethanolamine, dioleoylphosphatidylethanolamine (DOPE), dipalmitoyl phosphatidyl ethanolamine (DPPE), dimyristoylphosphoethanolamine (DMPE), distearoyl-phosphatidylethanolamine (DSPE), palmitoyloleoyl-phosphatidylethanolamine (POPE) palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleyolphosphatidylglycerol (POPG), 16-O-monomethyl PE, 16-O-dimethyl PE, 18-1-trans PE, palmitoyloleoyl-phosphatidylethanolamine (POPE), 1-stearoyl-2-oleoyl-phosphatidyethanolamine (SOPE), phosphatidylserine, phosphatidylinositol, sphingomyelin, cephalin, cardiolipin, phosphatidic acid, cerebrosides, dicetylphosphate, and cholesterol.

Additional nonphosphorous containing lipids that can be present in a lipid vesicle include stearylamine, dodecylamine, hexadecylamine, acetyl palmitate, glycerolricinoleate, hexadecyl stereate, isopropyl myristate, amphoteric acrylic polymers, triethanolamine-lauryl sulfate, alkyl-aryl sulfate polyethyloxylated fatty acid amides, dioctadecyldimethyl ammonium bromide and the like, diacylphosphatidylcholine, diacylphosphatidylethanolamine, ceramide, sphingomyelin, cephalin, and cerebrosides. Lipids such as lysophosphatidylcholine and lysophosphatidylethanolamine may also be present in the lipid vesicles described herein. Noncationic lipids also include polyethylene glycol-based polymers, such as PEG 2000, PEG 5000, and polyethylene glycol conjugated to phospholipids or to ceramides (referred to as PEG-Cer).

In some instances, modified forms of lipids may be used including forms modified with detectable labels such as fluorophores. In some instances, the lipid is a lipid analog that emits signal (e.g., a fluorescent signal). Examples include without limitation 1,1′-dioctadecyl-3,3,3′,3′-tetramethylindotricarbocyanine iodide (DiR) and 1,1′-dioctadecyl-3,3,3′,3′-tetramethylindodicarbocyanine (DiD).

In some examples of the lipid vesicles, at least one component of the lipid bilayer can be functionalized (or reactive) in order to allow for covalent attachment of the first binding partner. An example of a reactive group is a maleimide group. Maleimide groups may be crosslinked to each other in the presence of dithiol crosslinkers such as but not limited to dithiolthrietol (DTT). An example of a functionalized lipid is 1,2-dioleoyl-sn-glycero-3-phosphoethanolamine-N-[4-(p-maleimidop-henyl) butyramide, referred to herein as MPB. Another example of a functionalized lipid is 1,2-distearoyl-sn-glycero-3-phosphoethanolamine-N-[maleimide(polyethylene glycol)2000] (also referred to as maleimide-PEG 2k-PE). Another example of a functionalized lipid is dioleoyl-phosphatidylethanolamine 4-(N-maleimidomethyl)-cyclohexane-1-carboxylate (DOPE-mal).

It is to be understood that the invention contemplates the use of other functionalized lipids, other functionalized lipid bilayer components, other reactive groups, and other crosslinkers. In addition to the maleimide groups, other examples of reactive groups include but are not limited to other thiol reactive groups, amino groups such as primary and secondary amines, carboxyl groups, hydroxyl groups, aldehyde groups, alkyne groups, azide groups, carbonyls, haloacetyl (e.g., iodoacetyl) groups, imidoester groups, N-hydroxysuccinimide esters, sulfhydryl groups, pyridyl disulfide groups, and the like.

Functionalized and non-functionalized lipids are available from a number of commercial sources including Avanti Polar Lipids (Alabaster, Ala.).

In some embodiments, the lipid vesicles include a zwitterionic lipid molecule (e.g., poly(carboxybetaine) (pCB), poly(sulfobetaine)(pSB), or pDMAEMA).

In some embodiments, the lipid vesicles include polyelectrolyte multilayers (PEMs) or a polymer brush. Non-limiting examples of PEMs include poly-L-lysine/poly-L-glutamic acid (PLL/PLGA), poly-L-lysine/poly-L-glutamic acid. In some embodiments, the polymer brush includes [2-(acryloyloxy)ethylltrimethyl ammonium chloride (TMA), 2-carboxy ethyl acrylate (CAA). In some embodiments, the PEMs include one or more of: poly-L-lysine, poly-L-glutamic acid, and poly-L-aspartic acid. In some embodiments, the lipid vesicles include the polymer brush comprises [2-acryloyloxy)ethyl] trimethyl ammonium chloride (TMA) and 2-carboxyethyl acrylate (CAA).

In some aspects of any of the lipid vesicles described herein (e.g., non-fouling lipid vesicles), the lipid vesicle comprises 1-palmitoyl-2-oleoyl-sn-glycero-3-phosphocholine (POPC). In some aspects of any of the lipid vesicles described herein (e.g., non-fouling lipid vesicles), the lipid vesicle comprises 1,2-dipalmitoyl-sn-glycero-3-phosphoethanolamine-N-cap-biotinyl (b-PE). In some aspects of any of the lipid vesicles described herein (e.g., non-fouling lipid vesicles), the lipid vesicle comprises a combination of POPC and b-PE. For example, in any of the lipid vesicles described herein, the lipid vesicle can include a ratio of 200:1, 150:1, 100:1, 95:1, 90:1, 85:1, 80:1, 75:1, 70:1, 60:1, 50:1, 40:1, 30:1, 20:1, 10:1, 5:1, 200:5, 150:5, 100:5, 95:5, 90:5, 85:5, 80:5, 75:5, 70:5, 60:5, 50:5, 40:5, 30:5, 20:5, 10:5, 200:10, 150:10, 100:10, 90:10, 85:10, 80:10, 75:10, 70:10, 60:10, 50:10, 40:10, 30:10, 20:10, 200:15, 150:15, 100:15, 90:15, 85:15, 80:15, 75:15, 70:15, 60:15, 50:15, 40:15, 30:15, 20:15, 200:20, 150:20, 100:20, 95:20, 90:20, 85:20, 80:20, 70:20, 60:20, 50:20, 40:20, or 30:20) POPC/b-PE.

In some embodiments, the lipid vesicles include polyethylene glycol (PEG). In some embodiments, PEG exhibits a non-fouling property.

In some aspects of any of the lipid vesicles described herein (e.g., non-fouling lipid vesicles), the lipid vesicle is conjugated with a Dynabead™ biotin binder.

### First and Second Binding Partners

In some embodiments of any of the compositions described herein, the composition can include a plurality of first binding partners and a plurality of second binding partners. In some embodiments of any of the compositions described herein, each of the plurality of second binding partners is capable of specifically binding to one or more (e.g., two or more) (e.g., one, two, three, four, five, six, seven, eight, nine, or ten) first binding partners (e.g., capable of specifically binding to one or more (e.g., two or more) molecules of the same first binding partner), where a first subset of the plurality of the second binding partners specifically binds to (i) a first binding partner attached (covalently or non-covalently attached) to the exterior surface of the magnetic bead (e.g., any of the exemplary magnetic beads described herein or known in the art) and (ii) a first binding partner on the exterior surface of a lipid vesicle; a second subset of the plurality of the second binding partners specifically binds to (i) a first binding partner on the exterior surface of a lipid vesicle (e.g., the first binding partner attached either covalently or non-covalently to the exterior surface of the lipid vesicle), and (ii) a first binding partner attached to an agent that binds specifically to a target cell (e.g., any of the agents that bind specifically to a target cell described herein).

In some embodiments, the first binding partner is biotin or a variant thereof. In some embodiments, the first binding partner is streptavidin or a variant thereof. In some embodiments of any of the methods described herein, the first binding partner and the second binding partner can be interchanged. For example, the first binding partner can be biotin, or a derivative thereof, and the second binding partner is avidin, or a derivative thereof. In other examples, the first binding partner can be avidin, or a derivative thereof, and the second binding partner is biotin.

In some embodiments, a first binding partner can include an antigenic substance (e.g., a protein, a carbohydrate, a lipid, or a nucleic acid, or a combination thereof) and the second binding partner can include an antigen-binding domain (e.g., any of the exemplary antigen-binding domains described herein or known in the art) that binds specifically to the antigenic substance. In some embodiments, the first binding partner can include an antigen-binding domain (e.g., any of the exemplary antigen-binding domains described herein or known in the art) that binds specifically to an antigenic substance (e.g., a protein, a carbohydrate, a lipid, or a nucleic acid, or a combination thereof), and the second binding partner includes the antigenic substance.

In some embodiments, a first binding partner can include an aptamer that binds to a specific target moiety (e.g., a protein, a carbohydrate, a lipid, or a nucleic acid, or a combination thereof) and the second binding partner includes the specific target moiety. In some embodiments, a first binding partner can include a specific target moiety (e.g., a protein, a carbohydrate, a lipid, or a nucleic acid, or a combination thereof) and the second binding partner includes an aptamer that binds to the specific target moiety.

Additional examples of first binding partners and second binding partners are known in the art.

The first binding partner and the second binding partner provided herein can bind with a disassociation equilibrium constant (K_{D}) of less than 10⁻⁷ M, less than 10⁻⁸ M, less than 10⁻⁹M, less than 10⁻¹⁰ M, less than 10⁻¹¹ M, less than 10⁻¹² M, less than 10⁻¹³ M, less than 10⁻¹⁴ M, less than 10⁻¹⁵ M, or less than 10⁻¹⁶ M (e.g., as determined in phosphate buffered saline using surface plasmon resonance).

In some embodiments the first binding partner and the second binding partner provided herein can bind with a K_{D} of about 1×10⁻⁴ M to about 1×10⁻⁶ M, about 1×10⁻⁵M to about 1×10⁻⁷ M, about 1×10⁻⁶ M to about 1×10⁻⁸ M, about 1×10⁻⁷ M to about 1×10⁻⁹ M, about 1×10⁻⁸ M to about 1×10⁻¹⁰M, about 1×10⁻⁹ M to about 1×10⁻¹¹ M, about 1×10⁻⁹ M to about 1×10⁻¹²M, about 1×10⁻⁹M to about 1×10⁻¹³ M, about 1×10⁻⁹ M to about 1×10⁻¹⁴M, about 1×10⁻⁹M to about 1×10⁻¹⁵M, about 1×10⁻¹⁰M to about 1×10⁻¹⁵ M, about 1×10⁻¹⁰M to about 1×10⁻¹³M, about 1×10⁻¹³ M to about 1×10⁻¹⁵ M, or about 1×10⁻¹⁴ M to about 1×10⁻¹⁵ M (e.g., as determined in phosphate buffered saline using surface plasmon resonance). In some embodiments, the first binding partner and the second binding partner provided herein can bind with a K_{D} of about 1.1 nM to about 500 nM, or about 2.0 nM to about 6.7 nM.

In some embodiments of these compositions, the magnetic bead has covalently attached to its exterior surface the plurality of first binding partners (e.g., using any of the exemplary types of covalent bonds described herein). In some embodiments of these compositions, the magnetic bead has non-covalently attached to its exterior surface the plurality of first binding partners.

In some embodiments of these compositions, the plurality of agents that bind specifically to a target cell each comprise a covalently attached first binding partner (e.g., using any of the exemplary types of covalent bonds described herein). In some embodiments, the agent that specifically binds to a target cell includes an antigen-binding domain that binds to the target cell (e.g., an antigen present on the surface of the target cell). In some embodiments of these compositions, the plurality of agents that bind specifically to a target cell each include a non-covalently attached first binding partner.

Agents that Bind Specifically to a Target Cell

Provided herein are a plurality of agents that bind specifically to a target cell, wherein each agent includes an attached first binding partner (e.g., any of the exemplary first binding partners described herein or known in the art). Non-limiting examples of agents that can bind specifically to a target cell include: antibodies, antigen-binding antibody fragments, and aptamers. In some embodiments of these compositions, the plurality of agents that bind specifically to the target cell is an antibody or an antigen-binding fragment thereof.

As used herein, the term “antigen-binding domain” means a domain that is capable of specifically binding to an antigen (e.g., any of the exemplary antigens described herein). For example, an antigen-binding domain can be, e.g., a V_{L} domain, a V_{H} domain, a V_{NAR} domain, or a VIM domain.

An antigen-binding domain can also be, e.g., a non-antibody, scaffold protein. These proteins are, generally, obtained through combinatorial chemistry-based adaptation of preexisting antigen-binding proteins. For example, the binding site of human transferrin for human transferrin receptor can be diversified using the system described herein to create a diverse library of transferrin variants, some of which have acquired affinity for different antigens. See, e.g., Ali et al., J. Biol. Chem. 274:24066-24073, 1999. The portion of human transferrin not involved with binding the receptor remains unchanged and serves as a scaffold, like framework regions of antibodies, to present the variant binding sites. The libraries are then screened, as an antibody library is, and in accordance with the methods described herein, against a target antigen of interest to identify those variants having optimal selectivity and affinity for the target antigen. See, e.g., Hey et al., TRENDS Biotechnol. 23(10):514-522, 2005.

One of skill in the art would appreciate that the scaffold portion of the non-antibody scaffold protein can include, e.g., all or part of: the Z domain of S. aureus protein A, human transferrin, human tenth fibronectin type III domain, kunitz domain of a human trypsin inhibitor, human CTLA-4, an ankyrin repeat protein, a human lipocalin (e.g., anticalins, such as those described in, e.g., WO2015/104406), human crystallin, human ubiquitin, or a trypsin inhibitor from E. elaterium.

In some embodiments, the antigen-binding domain can be a scFv, a scFv-Fc, a VHH domain, a V_{NAR} domain, a (scFv)2, or a BiTE.

A “single-chain Fv’ or “scFv” fragment includes a V_{H} domain and a V_{L} domain in a single polypeptide chain. The V_{H} and V_{L} are generally linked by a peptide linker. See Pluckthun, Antibodies from E. coli. In Rosenberg M. & Moore G. P. (Eds.), The Pharmacology of Monoclonal Antibodies, Vol. 113, pp. 269-315, Spinger-Verlag, New York, 1994. In some examples, the linker can be a single amino acid. In some examples, the linker can be a chemical bond. “sc-Fv-Fc” fragments include an scFv attached to an Fc domain. For example, an Fc domain can be attached to the C-terminus of the scFv. The Fc domain can follow the V_{H} or V_{L}, depending on the orientation of the variable domains in the scFv (i.e., V_{H}-V_{L} or V_{L}-V_{H}). The Fc domain can be any suitable domain known in the art or described herein. In some examples, the Fc domain is an IgG1 Fc domain.

BiTEs are an antigen-binding domain that includes two V_{L} and two V_{H} in a single polypeptide that assemble to form two scFvs that recognize two different antigens or two different epitopes on a single antigen. Non-limiting aspects of BiTEs are described in Baeuerle et al., Curr. Opin. Mol. Ther 11:22-30, 2009; Wolf et al., Drug Discovery Today 10:1237-1244, 2005; and Huehls et al., Immunol. Cell Biol. 93:290-296, 2015.

A VHH domain is a single monomeric variable antibody domain found in camelids. A V_{NAR} domain is a single monomeric variable antibody domain found in cartilaginous fish. Non-limiting aspects of VHH domains and V_{NAR} domains are described in, e.g., Van Audenhove et al., EBioMedicine 8:40-48, 2016; Krah et al., Immunopharmacol. Immunotoxicol. 38:21-28, 2016; Cromie et al., Curr. Top. Med. Chem. 15:2543-2557, 2016; Kijanka et al., Nanomedicine 10:161-174, 2015; Kovaleva et al., Expert. Opin. Biol. Ther. 14:1527-1539, 2014; De Meyer et al., Trends Biotechnol. 32:263-270, 2014; Mujic-Delic et al., Trends Pharmacol. Sci. 35:247-255, 2014; Muyldermans, Ann. Rev. Biochem. 82:775-797, 2013; Vincke et al., Methods Mol. Biol. 911:15-26, 2012; Rahbarizadeh et al., Immunol. Invest. 40:299-338, 2011; Van Bockstaele et al., Curr. Opin. Investig. Drugs 10:1212-1224, 2009; Wesolowski et al., Med. Microbiol. Immunol. 198:157-174, 2009; De Genst et al., Dev. Comp. Immunol. 30:187-198, 2006; Muyldermans, J. Biotechnol. 74:277-302, 2001; and Muyldermans et al., Trends Biochem. Sci. 26:230-235, 2001.

In some embodiments, an antigen-binding domain can be an antigen-binding fragment of an antibody (e.g., any of the antigen-binding fragments of an antibody described herein), a DVD-Ig, and a dual-affinity re-targeting antibody (DART), a triomab, kih IgG with a common LC, a crossmab, an ortho-Fab IgG, a 2-in-1-IgG, IgG-ScFv, scFv₂-Fc, a bi-nanobody, tanden antibody, a DART-Fc, a scFv-HAS-scFv, DNL-Fab3, DAF (two-in-one or four-in-one), DutaMab, DT-IgG, knobs-in-holes common LC, knobs-in-holes assembly, charge pair antibody, Fab-arm exchange antibody, SEEDbody, Triomab, LUZ-Y, Fcab, kλ-body, orthogonal Fab, DVD-IgG, IgG(H)-scFv, scFv-(H)IgG, IgG(L)-scFv, scFv-(L)-IgG, IgG (L,H)-Fc, IgG(H)-V, V(H)—IgG, IgG(L)-V, V(L)-IgG, KIH IgG-scFab, 2scFv-IgG, IgG-2scFv, scFv4-Ig, Zybody, DVI-IgG, nanobody, nanobody-HSA, a diabody, a TandAb, scDiabody, scDiabody-CH3, Diabody-CH3, Triple Body, miniantibody, minibody, TriBi minibody, scFv-CH3 KIH, Fab-scFv, scFv-CH-CL-scFv, F(ab′)2-scFV₂, scFv-KIH, Fab-scFv-Fc, tetravalent HCAb, scDiabody-Fc, diabody-Fc, tandem scFv-Fc, intrabody, dock and lock bispecific antibody, ImmTAC, HSAbody, scDiabody-HAS, tandem scFv, IgG-IgG, Cov-X-Body, and scFv1-PEG-scFv2. Non-limiting examples of an antigen-binding fragment of an antibody include an Fv fragment, a Fab fragment, a F(ab′)₂ fragment, and a Fab′ fragment. Additional examples of an antigen-binding fragment of an antibody is an antigen-binding fragment of an IgG (e.g., an antigen-binding fragment of IgG1, IgG2, IgG3, or IgG4) (e.g., an antigen-binding fragment of a human or humanized IgG, e.g., human or humanized IgG1, IgG2, IgG3, or IgG4); an antigen-binding fragment of an IgA (e.g., an antigen-binding fragment of IgA1 or IgA2) (e.g., an antigen-binding fragment of a human or humanized IgA, e.g., a human or humanized IgA1 or IgA2); an antigen-binding fragment of an IgD (e.g., an antigen-binding fragment of a human or humanized IgD); an antigen-binding fragment of an IgE (e.g., an antigen-binding fragment of a human or humanized IgE); or an antigen-binding fragment of an IgM (e.g., an antigen-binding fragment of a human or humanized IgM).

A “Fv” fragment includes a non-covalently-linked dimer of one heavy chain variable domain and one light chain variable domain.

A “Fab” fragment includes, in addition to the heavy and light chain variable domains of the Fv fragment, the constant domain of the light chain and the first constant domain (Cm) of the heavy chain.

A “F(ab′)₂” fragment includes two Fab fragments joined, near the hinge region, by disulfide bonds.

A “dual variable domain immunoglobulin” or “DVD-Ig” refers to multivalent and multispecific binding proteins as described, e.g., in DiGiammarino et al., Methods Mol. Biol. 899:145-156, 2012; Jakob et al., MABs 5:358-363, 2013; and U.S. Pat. Nos. 7,612,181; 8,258,268; 8,586,714; 8,716,450; 8,722,855; 8,735,546; and 8,822,645, each of which is incorporated by reference in its entirety.

DARTs are described in, e.g., Garber, Nature Reviews Drug Discovery 13:799-801, 2014. A description of a triomabs, kih IgG with a common LCs, crossmabs, ortho-Fab IgGs, 2-in-1-IgGs, IgG-ScFvs, scFv₂-Fcs, bi-nanobodies, tanden antibodies, DART-Fcs, scFv-HAS-scFvs, and DNL-Fab3s are described in, e.g., Kontermann et al., Drug Discovery Today 20:838-847, 2015. A description of DAFs (two-in-one or four-in-one), DutaMabs, DT-IgGs, knobs-in-holes common LCs, knobs-in-holes assemblies, charge pair antibodies, Fab-arm exchange antibodies, SEEDbodies, Triomabs, LUZ-Ys, Fcabs, la-bodies, orthogonal Fabs, DVD-IgGs, IgG(H)-scFvs, scFv-(H)IgGs, IgG(L)-scFvs, scFv-(L)-IgGs, IgG (L,H)-Fcs, IgG(H)-Vs, V(H)-IgGs, IgG(L)-Vs, V(L)-IgGs, KIH IgG-scFabs, 2scFv-IgGs, IgG-2scFvs, scFv4-Igs, Zybodies, DVI-IgGs, nanobodies, nanobody-HSAs, a diabodies, a TandAbs, scDiabodies, scDiabody-CH3s, Diabody-CH3s, Triple Bodies, miniantibodies, minibodies, TriBi minibodies, scFv-CH3 KIHs, Fab-scFvs, scFv-CH-CL-scFvs, F(ab′)₂-scFV₂s, scFv-KIHs, Fab-scFv-Fcs, tetravalent HCAbs, scDiabody-Fcs, diabody-Fcs, tandem scFv-Fcs, intrabodies, dock and lock bispecific antibodies, ImmTACs, HSAbodies, scDiabody-HASs, tandem scFvs, IgG-IgGs, Cov-X-Bodies, and scFv1-PEG-scFv2s are described in, e.g., Spiess et al., Mol. Immunol. 67:95-106, 2015.

In some embodiments of the compositions described herein, the plurality of agents that bind specifically to the target cell (e.g., any of the exemplary target cells described herein) is an antibody or an antigen-binding fragment thereof that specifically binds to a cancer antigen (e.g., any of the exemplary cancer antigens described herein). In some embodiments of these compositions, the cancer antigen is epithelial cell adhesion molecule (EpCAM). Additional examples of cancer antigens include HER2, A33 antigen, 9-0-acetyl-GD3, CA19-9 marker, BhCG, CA-125 marker, carboanhydrase IX (MN/CA IX), calreticulin, CCR5, CCR8, CD2, CD3, CDS, CD16, CD19, CD20, CD22, CD24, CD25, CD27, CD28, CD30, CD33, CD38, CD40L, CD44, CD44V6, CD63, CD70, CD84, CD96, CD100, CC123, CD133, CD137, CD138, CD150, CD152 (CTLA-4), CD160, CRTAM, CS1 (CD319), DNAM-1 (CD226), CD229, CD244, CD272 (BTLA), CD274 (PDL-1, B7H1), CD279 (PD-1), CD319, CD352, CRTAM (CD355), CD358, DR3, GITR (TNFRSF 18), HVEM, ICOS, LIGHT, LTBR, OX40, activating forms of KIR, NKG2C, NKG2D, NKG2E, NTB-A, PEN-5, carcinoma embryonic antigen (CEA; CD66e), desmoglein 4, E-cadherin neoepitope, endosialin, ephrin A2 (EphA2), epidermal growth factor receptor (EGFR), epithelial cell adhesion molecule (EpCAM), fucosyl GM1, GD2, GD3, GM2, ganglioside GM3, Globo H, glycoprotein 100, HER2/neu, HER3, HER4, insulin-like growth factor receptor 1, Lewis-Y, LG, Ly-6, melanoma-specific chondroitin-sulfate proteoglycan (MCSCP), mesothelin, MUC1, MUC2, MUC3, MUC4, MUC5AC, MUC5b, MUC7, MUC16, Mullerian inhibitory substance (MIS) receptor type II, plasma cell antigen, poly SA, PSCA, PSMA, sonic hedgehog (SHH), SAS, STEAP, sTn antigen, TNF-alpha precursor, 2B4 (CD244), β2-integrins, KIR, KIR2DL1, KIR2DL2, KIR2DL3, KIR3DL2, KIR-L, KLRGI, LAIR-1, NKG2A, NKR-P IA, Siglec-3, Siglec-7, Siglec-9, TCRa, TCRB, TCRSy, TIM1, LAG3, LAIR1, PD-1H, TIGIT, TIM2, and TIM3.

### Target Cells

The compositions and methods described herein can be used to capture and isolate target cells. Non-limiting examples of target cells include: cancer cells (e.g., circulating cancer cells), immune cells (e.g., T-cells, B-cells, macrophages, neutrophils, or dendritic cells), bacterial cells, virus-infected cells, stem cells (e.g., bone marrow stem cells), fetal cells, and epithelial cells.

In some embodiments, the target cell is a eukaryotic cell (e.g., a mammalian cell), or a prokaryotic cell.

Non-limiting examples of cancer include: acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, Burkitt Lymphoma, carcinoma of unknown primary origin, cardiac tumor, cervical cancer, chordoma, chronic lymphocytic leukemia (CLL), chronic myelogenous leukemia (CML), chronic myeloproliferative neoplasm, colon cancer, colorectal cancer, craniopharyngioma, cutaneous T-cell lymphoma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hairy cell leukemia, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, leukemia, lip and oral cavity cancer, liver cancer, lobular carcinoma in situ, lung cancer, lymphoma, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia, syndrome, multiple myeloma, mycosis fungoides, myelodysplastic syndrome, myelodysplastic/myeloproliferative neoplasm, nasal cavity and para-nasal sinus cancer, nasopharyngeal cancer, neuroblastoma, non-Hodgkin lymphoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms' tumor. The cancer cells can, e.g., be derived from a subject identified or diagnosed as having any of the cancers described herein. The cancer cells, e.g., can be derived from a subject suspected of having any of the cancers described herein.

For example, a target cell can be selected from the group consisting of: melanoma cells, breast cancer cells, lung cancer cells, bladder cancer cells, colon cancer cells, pancreatic cancer cells, stomach cancer cells, and uterine cancer cells.

### Kits

Also provided herein are kits containing one or more (e.g., at least 2, 3, 4, 5, 6, 7, 8, 9, 10, 12, 14, 16, 18, or 20) of any of the compositions described herein. In some embodiments, the kits can include instructions for performing any of the methods described herein. In some embodiments, the kits can include a volume of a culture medium for culturing the target cell (e.g., a culture medium that can further include a selection agent, e.g., an antibiotic).

### Methods of Generating a Magnetic Bead

Also provided herein are methods of generating a magnetic bead having attached to its exterior surface a plurality of vesicles that include: (a) applying a magnetic field to a composition that includes: (i) a magnetic bead (e.g., any of the magnetic beads described herein or known in the art) having attached to its exterior surface (e.g., either covalently or non-covalently attached to its exterior surface) a plurality of first binding partners (e.g., any of the first binding partners described herein or known in the art); (ii) a plurality of lipid vesicles (e.g., any of the lipid vesicles described herein or known in the art) that comprise a plurality of the first binding partners on its exterior surface (e.g., the first binding partners being either covalently or non-covalently attached to its exterior surface); (iii) a plurality of second binding partners (e.g., any of the exemplary second binding partners described herein or known in the art); and (iv) a plurality of agents that bind specifically to a target cell (e.g., any of the exemplary agents that bind specifically to a target cell described herein or known in the art), where each agent comprises an attached first binding partner; wherein: each of the plurality of second binding partners is capable of specifically binding to one or more (e.g., two or more) (e.g., one, two, three, four, five, six, seven, eight, nine, or ten) first binding partners, a first subset of the plurality of the second binding partners specifically binds to (i) a first binding partner attached to the exterior surface of the magnetic bead and (ii) a first binding partner on the exterior surface of a lipid vesicle; a second subset of the plurality of the second binding partners specifically binds to (i) a first binding partner on the exterior surface of a lipid vesicle, and (ii) a first binding partner attached to an agent that binds specifically to a target cell, where the magnetic field is applied under conditions sufficient to allow the association between (i) the first binding partner and the second binding partner; (b) after step (a), washing the magnetic bead with a wash buffer under conditions sufficient to allow the association between the first binding partner and the second binding partner; and (c) after step (b), resuspending the washed beads with an aqueous solution (e.g., an aqueous solution including between 1% and 10% bovine serum albumin) under conditions that allow the association between the first binding partner and the second binding partner, thereby generating a magnetic bead having attached to its exterior surface a plurality of lipid vesicles.

In some embodiments of any of the methods described herein, the method includes a step of applying a magnetic force. In some embodiments, magnetic force is applied using a magnet or a device including a magnet, a magnetic bar, a magnetic stand (e.g., Ambion® single tube magnetic stand), or a magnetic separation rack (e.g., New England BioLabs® 12-tube magnetic separation rack). Additional exemplary methods for applying a magnetic force are known in the art.

In some embodiments of any of the methods described herein, the method can further include at least one (e.g., 2, 3, 4, 5, 6, 7, or 8) washing steps after the contacting step. In some embodiments of any of the methods described herein, the method can further include at least four (e.g., 5, 6, 7, 8, 9, 10, 11 or 12) washing steps after the contacting step.

In some embodiments of any of the methods described herein, the at least one washing step includes the use of a wash buffer (e.g., any of the wash buffers described herein). In some embodiments, the washing step includes use of a wash buffer (e.g., any of the exemplary wash buffers described herein) at a temperature of about 10° C. to about 37° C. (e.g., about 10° C. to about 35° C., about 10° C. to about 30° C., about 10° C. to about 28° C., about 10° C. to about 26° C., about 10° C. to about 24° C., about 10° C. to about 22° C., about 10° C. to about 20° C., about 10° C. to about 18° C., about 10° C. to about 16° C., about 10° C. to about 14° C., about 10° C. to about 12° C., about 12° C. to about 37° C., about 12° C. to about 35° C., about 12° C. to about 30° C., about 12° C. to about 28° C., about 12° C. to about 26° C., about 12° C. to about 24° C., about 12° C. to about 22° C., about 12° C. to about 20° C., about 12° C. to about 18° C., about 12° C. to about 16° C., about 12° C. to about 14° C., about 14° C. to about 37° C., about 14° C. to about 35° C., about 14° C. to about 30° C., about 14° C. to about 28° C., about 14° C. to about 26° C., about 14° C. to about 24° C., about 14° C. to about 22° C., about 14° C. to about 20° C., about 14° C. to about 18° C., about 14° C. to about 16° C., about 16° C. to about 37° C., about 16° C. to about 35° C., about 16° C. to about 30° C., about 16° C. to about 28° C., about 16° C. to about 26° C., about 16° C. to about 24° C., about 16° C. to about 22° C., about 16° C. to about 20° C., about 16° C. to about 18° C., about 18° C. to about 37° C., about 18° C. to about 35° C., about 18° C. to about 30° C., about 18° C. to about 28° C., about 18° C. to about 26° C., about 18° C. to about 24° C., about 18° C. to about 22° C., about 18° C. to about 20° C., about 20° C. to about 37° C., about 20° C. to about 35° C., about 20° C. to about 30° C., about 20° C. to about 28° C., about 20° C. to about 26° C., about 20° C. to about 24° C., about 20° C. to about 22° C., about 22° C. to about 37° C., about 22° C. to about 35° C., about 22° C. to about 30° C., about 22° C. to about 28° C., about 22° C. to about 26° C., about 22° C. to about 24° C., about 24° C. to about 37° C., about 24° C. to about 35° C., about 24° C. to about 28° C., about 24° C. to about 26° C., about 26° C. to about 37° C., about 26° C. to about 35° C., about 26° C. to about 30° C., about 26° C. to about 28° C., about 28° C. to about 37° C., about 28° C. to about 35° C., about 28° C. to about 30° C., about 30° C. to about 37° C., about 30° C. to about 35° C., or about 35° C. to about 37° C.) for about 10 seconds to about 6 hours (e.g., about 10 seconds to about 5 hours, about 10 seconds to about 4 hours, about 10 seconds to about 3 hours, about 10 seconds to about 2 hours, about 10 seconds to about 1 hour, about 10 seconds to about 50 minutes, about 10 seconds to about 40 minutes, about 10 seconds to about 30 minutes, about 10 seconds to about 20 minutes, about 10 seconds to about 15 minutes, about 10 seconds to about 10 minutes, about 10 seconds to about 5 minutes, about 10 seconds to about 1 minute, about 10 seconds to about 30 seconds, about 30 seconds to about 6 hours, about 30 seconds to about 5 hours, about 30 seconds to about 4 hours, about 30 seconds to about 3 hours, about 30 seconds to about 2 hours, about 30 seconds to about 1 hour, about 30 seconds to about 50 minutes, about 30 seconds to about 40 minutes, about 30 seconds to about 30 minutes, about 30 seconds to about 20 minutes, about 30 seconds to about 15 minutes, about 30 seconds to about 10 minutes, about 30 seconds to about 5 minutes, about 30 seconds to about 1 minute, about 1 minute to about 6 hours, about 1 minute to about 5 hours, about 1 minute to about 4 hours, about 1 minute to about 3 hours, about 1 minute to about 2 hours, about 1 minute to about 1 hour, about 1 minute to about 50 minutes, about 1 minute to about 40 minutes, about 1 minute to about 30 minutes, about 1 minute to about 20 minutes, about 1 minute to about 15 minutes, about 1 minute to about 10 minutes, about 1 minute to about 5 minutes, about 5 minutes to about 6 hours, about 5 minutes to about 5 hours, about 5 minutes to about 4 hours, about 5 minutes to about 3 hours, about 5 minutes to about 2 hours, about 5 minutes to about 1 hour, about 5 minutes to about 50 minutes, about 5 minutes to about 40 minutes, about 5 minutes to about 30 minutes, about 5 minutes to about 20 minutes, about 5 minutes to about 15 minutes, about 5 minutes to about 10 minutes, about 10 minutes to about 6 hours, about 10 minutes to about 5 hours, about 10 minutes to about 4 hours, about 10 minutes to about 3 hours, about 10 minutes to about 2 hours, about 10 minutes to about 1 hour, about 10 minutes to about 50 minutes, about 10 minutes to about 40 minutes, about 10 minutes to about 30 minutes, about 10 minutes to about 20 minutes, about 10 minutes to about 15 minutes, about 15 minutes to about 6 hours, about 15 minutes to about 5 hours, about 15 minutes to about 4 hours, about 15 minutes to about 3 hours, about 15 minutes to about 2 hours, about 15 minutes to about 1 hour, about 15 minutes to about 50 minutes, about 15 minutes to about 40 minutes, about 15 minutes to about 30 minutes, about 15 minutes to about 20 minutes, about 20 minutes to about 6 hours, about 20 minutes to about 5 hours, about 20 minutes to about 4 hours, about 20 minutes to about 3 hours, about 20 minutes to about 2 hours, about 20 minutes to about 1 hour, about 20 minutes to about 50 minutes, about 20 minutes to about 40 minutes, about 20 minutes to about 30 minutes, about 30 minutes to about 6 hours, about 30 minutes to about 5 hours, about 30 minutes to about 4 hours, about 30 minutes to about 3 hours, about 30 minutes to about 2 hours, about 30 minutes to about 1 hour, about 30 minutes to about 50 minutes, about 30 minutes to about 40 minutes, about 40 minutes to about 6 hours, about 40 minutes to about 5 hours, about 40 minutes to about 4 hours, about 40 minutes to about 3 hours, about 40 minutes to about 2 hours, about 40 minutes to about 1 hour, about 40 minutes to about 50 minutes, about 50 minutes to about 6 hours, about 50 minutes to about 5 hours, about 50 minutes to about 4 hours, about 50 minutes to about 3 hours, about 50 minutes to about 2 hours, about 50 minutes to about 1 hour, about 1 hour to about 6 hours, about 1 hour to about 5 hours, about 1 hour to about 4 hours, about 1 hour to about 3 hours, about 1 hour to about 2 hours, about 2 hours to about 6 hours, about 2 hours to about 5 hours, about 2 hours to about 4 hours, about 2 hours to about 3 hours, about 3 hours to about 6 hours, about 3 hours to about 5 hours, about 3 hours to about 4 hours, about 4 hours to about 6 hours, about 4 hours to about 5 hours, or about 5 hours to about 6 hours).

In some embodiments, the wash buffer includes phosphate buffered saline (PBS), and bovine serum albumin (BSA) or serum (e.g., fetal calf serum or normal goat serum (GS)). In some embodiments of any of the wash buffers described herein, the wash buffer includes about 0.1% w/v to about 10% w/v (e.g., about 0.1% w/v to about 5% w/v, about 0.1% w/v to about 1% w/v, about 0.1 w/v to about 0.5% w/v, about 0.5% w/v to about 10% w/v, about 0.5% w/v to about 5% w/v, about 0.5% w/v to about 1% w/v, about 1% w/v to about 10% w/v, about 1% w/v to about 5% w/v, about 1% w/v to about 2% w/v, about 2% w/v to about 10% w/v, about 2% w/v to about 5% w/v; about 0.1% w/v, about 0.5% w/v, about 1% w/v, about 2% w/v, about 5% w/v, or about 10% w/v) BSA or serum (e.g., fetal calf serum or normal goat serum).

In some embodiments of any of the methods described herein, the method can further include resuspending the washed beads with an aqueous solution (e.g., any of the aqueous solutions described herein, e.g., an aqueous solution that includes between about 1% w/v to about 10% w/v, about 1% w/v to about 5% w/v, about 1% w/v to about 2% w/v, about 2% w/v to about 10% w/v, about 2% w/v to about 5% w/v; about 1% w/v, about 2% w/v, about 3% w/v, about 4% w/v, about 5% w/v, about 6% w/v, about 7% w/v, about 8% w/v, about 9% w/v, or about 10% w/v BSA or serum (e.g., fetal calf serum, normal goat serum)). In some embodiments, the resuspension of the washed beads with an aqueous solution results in a homogeneously dispersion of the washed beads with little to no visible aggregation.

In some embodiments of any of the methods described herein, the method can further include resuspending the washed beads in about 1 μL to about 500 mL of (e.g., about 1 μL to about 200 mL, about 1 μL to about 100 mL, about 1 μL to about 50 mL, about 1 μL to about 10 mL, about 1 μL to about 5 mL, about 1 μL to about 1 mL, about 1 μL to about 500 μL, about 1 μL to about 100 μL, about 1 μL to about 50 μL, about 1 μL to about 20 μL, about 100 μL to about 500 mL, about 100 μL to about 200 mL, about 100 μL to about 100 mL, about 100 μL to about 50 mL, about 100 μL to about 10 mL, about 100 μL to about 5 mL, about 100 μL to about 1 mL, about 100 μL to about 500 μL, about 1 mL to about 500 mL, about 1 mL to about 200 mL, about 1 mL to about 100 mL, about 1 mL to about 50 mL, about 1 mL to about 20 mL, about 1 mL to about 10 mL, about 1 mL to about 5 mL, about 5 mL to about 500 mL, about 5 mL to about 100 mL, about 5 mL to about 50 mL, about 5 mL to about 20 mL, about 10 mL to about 500 mL, about 10 mL to about 200 mL, about 10 mL to about 100 mL, about 10 mL to about 50 mL, about 10 mL to about 20 mL, about 50 mL to about 500 mL, about 50 mL to about 200 mL, about 50 mL to about 100 mL, about 100 mL to about 500 mL, about 100 mL to about 200 mL, or about 200 mL to about 500 mL) an aqueous solution (e.g., any of the aqueous solutions described herein).

Also provided herein are methods of generating a magnetic bead having attached to its exterior surface a plurality of lipid vesicles that include: (a) incubating: (i) a magnetic bead (e.g., any of the exemplary magnetic beads described herein or known in the art) having attached (e.g., covalently or non-covalently attached) to its exterior surface a plurality of first binding partners (e.g., any of the first binding partners described herein or known in the art); (ii) a plurality of lipid vesicles (e.g., any of the exemplary lipid vesicles described herein or known in the art) that include a plurality of the first binding partners on its exterior surface (e.g., covalently or non-covalently attached to its exterior surface); and (iii) a plurality of second binding partners (e.g., any of the second binding partners described herein or known in the art); wherein: each of the plurality of second binding partners is capable of specifically binding to one or more (e.g., two or more) first binding partners, a subset of the plurality of the second binding partners specifically binds to (i) a first binding partner attached to the exterior surface of the magnetic bead and (ii) a first binding partner on the exterior surface of a lipid vesicle; under conditions sufficient to allow the association between (i) the first binding partner and the second binding partner; (b) after step (a), washing the magnetic bead with a wash buffer under conditions sufficient to allow the association between the first binding partner and the second binding partner; (c) after (b), contacting the magnetic bead with a plurality of agents that bind specifically to a target cell, wherein each agent comprises an attached first binding partner, under conditions sufficient to allow the association between the first binding partner and the second binding partner, thereby generating a magnetic bead having attached to its exterior surface a plurality of lipid vesicles.

In some embodiments of any of the methods described herein, the incubating step is performed at a temperature of about 10° C. to about 37° C. (or any of the subranges of this range described herein). In some embodiments of any of the methods described herein, the incubating step is performed for about 10 seconds to about 6 hours (or any of the subranges of this range described herein). In some embodiments, the washing step can be performed using any of the exemplary aqueous buffers described herein.

In some embodiments of any of the methods described herein, the method can further include at least one (e.g., 2, 3, 4, 5, 6, 7, or 8) washing steps after the incubating step.

In some embodiments of any of the methods described herein, the at least one washing step includes the use of a wash buffer (e.g., any of the wash buffers described herein). In some embodiments, the washing step includes use of a wash buffer (e.g., any of the exemplary wash buffers described herein) at a temperature of about 10° C. to about 37° C. (or any of the subranges of this range described herein) for about 1 minute to about 6 hours (or any of the subranges of this range described herein).

In some embodiments, the wash buffer includes phosphate buffered saline (PBS) and optionally includes bovine serum albumin (BSA) or serum (e.g., fetal calf serum or normal goat serum). In some embodiments of any of the wash buffers described herein, the wash buffer includes about 0.1% w/v to about 10% w/v (or any of the subranges of this range described herein) BSA or serum (e.g., fetal calf serum or normal goat serum).

In some embodiments of any of the methods described herein, the contacting of the magnetic bead with a plurality of agents that bind specifically to a target cell is performed at a temperature of about 10° C. to about 37° C. (or any of the subranges of this range described herein). In some embodiments of any of the methods described herein, the contacting step is performed for about 10 seconds to about 6 hours (or any of the subranges of this range described herein). The contacting step can be performed using any of the aqueous buffers described here.

### Methods of Isolating a Target Cell

Provided herein are methods of isolating a target cell (e.g., any of the exemplary target cells described herein or known in the art) from a biological sample (e.g., a biological sample including blood, serum, or plasma) that include: (a) contacting a biological sample comprising a target cell and non-target cells with any of the compositions described herein; (b) after (a), washing the magnetic bead with a wash buffer under conditions sufficient to allow the association between (i) the first binding partner and the second binding partner to form a complex, and (ii) the agent that binds specifically to the target cell and the complex; and (c) after (b), applying a magnetic force to the magnetic bead under conditions sufficient to allow the association between (i) the first binding partner and the second binding partner, and (ii) the target cell and the agent that binds specifically to the target cell, thereby isolating the target cell.

In some embodiments of any of the methods described herein, the contacting of a biological sample is performed at a temperature of about 10° C. to about 37° C. (or any of the subranges of this range described herein). In some embodiments of any of the methods described herein, the contacting step is performed for about 10 seconds to about 6 hours (or any of the subranges of this range described herein). The contacting step can be performed using any of the exemplary aqueous buffers described herein.

### Washing

In some embodiments of any of the methods described herein, the method can further include at least one (e.g., 2, 3, 4, 5, 6, 7, or 8) washing steps after the contacting step. In some embodiments of any of the methods described herein, the at least one washing step includes the use of a wash buffer (e.g., any of the exemplary wash buffers described herein). In some embodiments, the washing step includes use of a wash buffer (e.g., any of the exemplary wash buffers described herein) at a temperature of about 10° C. to about 37° C. (or any of the subranges of this range described herein) for about 10 seconds to about 6 hours (or any of the subranges of this range described herein).

In some embodiments, the wash buffer includes phosphate buffered saline (PBS) and optionally, bovine serum albumin (BSA) or serum (e.g., fetal calf serum or normal goat serum). In some embodiments of any of the wash buffers described herein, the wash buffer includes about 0.1% w/v to about 10% w/v (or any of the subranges of this range described herein) BSA or serum (e.g., fetal calf serum or normal goat serum).

### Applying a Magnetic Force

In some embodiments of any of the methods described herein, the method includes a step of applying a magnetic force. In some embodiments, magnetic force is applied using a magnet or a device including a magnet, a magnetic bar, a magnetic stand (e.g., Ambion® single tube magnetic stand), or a magnetic separation rack (e.g., New England BioLabs® 12-tube magnetic separation rack). Additional exemplary methods for applying a magnetic force are known in the art.

### One or More Additional Steps

In some embodiments of any of the methods described herein, one or more additional steps can be performed before and/or after the step of applying a magnetic force.

In some embodiments, the one or more (e.g., two, three, four or five) additional steps performed before the applying the magnetic force step can include: lysing red blood cells in the sample. Red blood cell lysis can be performed by incubating the sample with a red blood cell lysis buffer (e.g., 155 mM NH₄C1, 10 mM KHCO₃, 0.1 mM EDTA, pH 7.3). In some embodiments, the red blood cell lysis buffer includes ammonium chloride and potassium bicarbonate, and optionally ethylenediaminetetraacetic acid (EDTA).

In some embodiments, the one or more (e.g., two, three, four or five) additional steps performed after the applying the magnetic force step include: culturing the target cells, quantifying the target cells, determining the cell viability of the target cells, staining the target cells (e.g., immunostaining the target cells), genetically modifying the target cells, injecting the target cells into a subject, performing an in vitro assay using the target cells, freezing the target cells, extracting and optionally sequencing nucleic acids obtained from the target cells, and selecting and/or administering a pharmaceutical treatment to a subject based specifically on the detected genotype of the nucleic acid extracted from the target cells.

In some embodiments, the one or more additional steps performed before applying the magnetic force include lysing red blood cells, and the one or more additional steps performed after the magnetic force is applied is selected from the group of: culturing the target cells, quantifying the target cells, determining the cell viability of the target cells, extracting nucleic acids from the target cells, and genotyping the nucleic acid extracted from the targets cells.

In some embodiments, the one or more additional steps performed before applying the magnetic force include lysing red blood cells; and the one or more additional steps performed after the magnetic force is applied is selected from the group of: culturing the target cells, quantifying the target cells, determining the cell viability of the target cells, extracting nucleic acids from the target cells, genotyping the nucleic acid extracted from the targets cells, and selecting and/or administering a pharmaceutical treatment to a subject based specifically on the genotype of the nucleic acid extracted from the target cells.

In some embodiments, the one or more additional steps performed before applying the magnetic force include lysing red blood cells; and the one or more additional steps performed after the magnetic force is applied is selected from the group of: culturing the target cells, quantifying the target cells, determining the cell viability of the target cells, and freezing the target cells.

In some embodiments, the one or more additional steps performed before applying the magnetic force include lysing red blood cells; and the one or more additional steps performed after the magnetic force is applied is selected from the group of: culturing the target cells, quantifying the target cells, determining the cell viability of the target cells, and genetically modifying the target cells and optionally, freezing the genetically-modified target cells.

In some embodiments, the one or more additional steps performed before applying the magnetic force include lysing red blood cells; and the one or more additional steps performed after the magnetic force is applied is selected from the group of: culturing the target cells, quantifying the target cells, determining the cell viability of the target cells, and genetically modifying the target cells, and optionally, injecting the genetically-modified target cells into a subject.

In some embodiments, the one or more additional steps performed before applying the magnetic force include lysing red blood cells; and the one or more additional steps performed after the magnetic force is applied is selected from the group of: culturing the target cells, quantifying the target cells, determining the cell viability of the target cells, and genetically modifying the target cells and optionally, freezing the genetically-modified target cells.

A variety of different methods known in the art can be used to genetically modify a target cell. Non-limiting examples of methods that can be used to genetically modify a target cell include transformation, lipofection, transfection, electroporation, microinjection, calcium phosphate transfection, dendrimer-based transfection, cationic polymer transfection, cell squeezing, optical transfection, hydrodynamic delivery, viral transduction (e.g., adenoviral and lentiviral transduction), and nanoparticle transfection. These and other methods of genetically modifying a target cell are well known in the art.

Various methods of identifying and detecting a target cell are known in the art, such methods include, but are not limited to, flow cytometry, e.g., fluorescence-assisted cell sorting (FACS), ELISA, Western blot analysis, immunoprecipitation, protein microarrays, immunofluorescence, Sanger sequencing method, Maxam-Gilbert sequencing method, capillary electrophoresis, pyrosequencing, single-molecule real-time sequencing, and many others known in the art.

Various methods of culturing, quantifying and determining cell viability of a cell (e.g., a target cell) are known in the art, and may be used in any of the methods described herein.

### EXAMPLES

The invention is further described in the following examples, which do not limit the scope of the invention described in the claims.

### Example 1. Exemplary Circulating Tumor Cell Capture Protocol

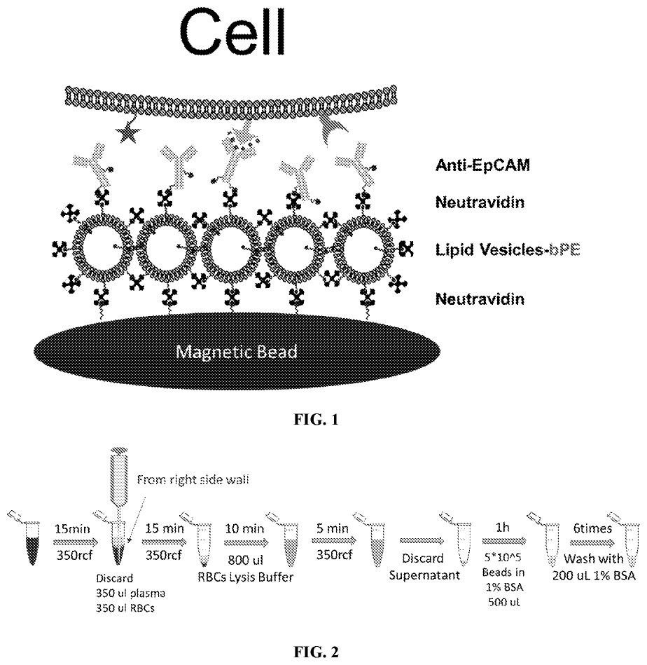

The lipid vesicle coated magnetic beads described herein were used to isolate circulating tumor cells (CTCs) from a biological sample. Briefly, 1.25 mL of blood is disposed in a 1.5 mL-Eppendorf tube. The Eppendorf tube is then centrifuged for 15 minutes at 350 relative centrifugal speed (rcf). Next, the supernatant was discarded (e.g., 350 μL of plasma and 350 μL of red blood cells) and the cell pellet was kept. The cell pellet was then re-suspended in 800 μL of red blood cell lysis buffer (155 mM NH₄ Cl, 10 mM KHCO₃, 0.1 mM EDTA, pH 7.3) and incubated for 15 minutes at room temperature. The sample was then centrifuged for 5 minutes at 350 rcf, and the supernatant was discarded. Next, the pellet was resuspended in 500 μL of a solution that included 1% bovine serum albumin (BSA), and 5 μL of CMx beads were added (1.0×10⁵ beads/mL). The Eppendorf tube was then placed onto a rotator and incubated for 1 hour at room temperature. Following the incubation, the Eppendorf tube was spun for approximately 3-5 seconds to prevent liquid adsorption on the cap. The tube was then placed onto a magnet for 1 minute, after which the supernatant was discarded. The tube was then washed six times with 200 μL with a solution that included 1× phosphate buffered saline (PBS) (pH 7.0) and 1% BSA for 5 minutes. The sample contained within the tube was then ready for further downstream analysis or was stored at −80° C. A schematic representation of an exemplary embodiment of a composition is shown in FIG. 1. A flow chart of the protocol is shown in FIG. 2.

### Example 2. Target Cell Recovery Rate and Purity from Whole Blood

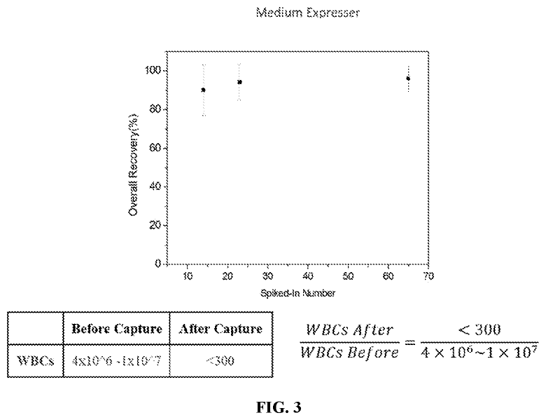

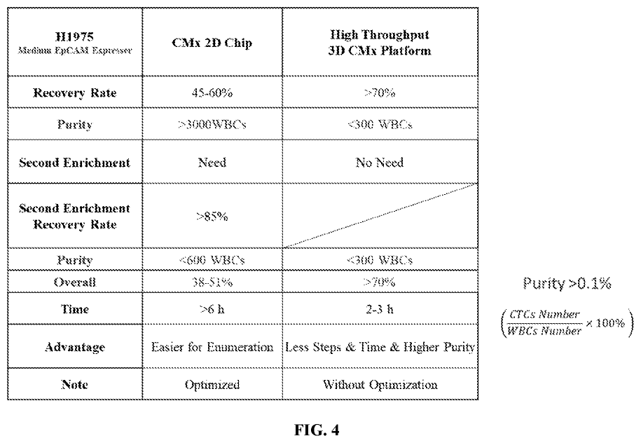

The circulating tumor cell capture protocol was applied to medium EpCAM-expressing cell (H1975)s. The data from this experiment is shown in FIG. 3. The data show a very good recovery rate of the target cell (H1975) from whole blood when the lipid vesicle-coated magnetic beads provided herein are used. The recovery rate can be higher than 70% in experiments where the lipid vesicle-coated magnetic beads are exposed to three different concentrations of cells (i.e., three different cell samples including different total concentrations of cells). The use of the lipid vesicle-coated magnetic beads provided herein also results in a reduced level of residual white blood cells (a level of equal to or less than 300 white blood cells with six times of washing).

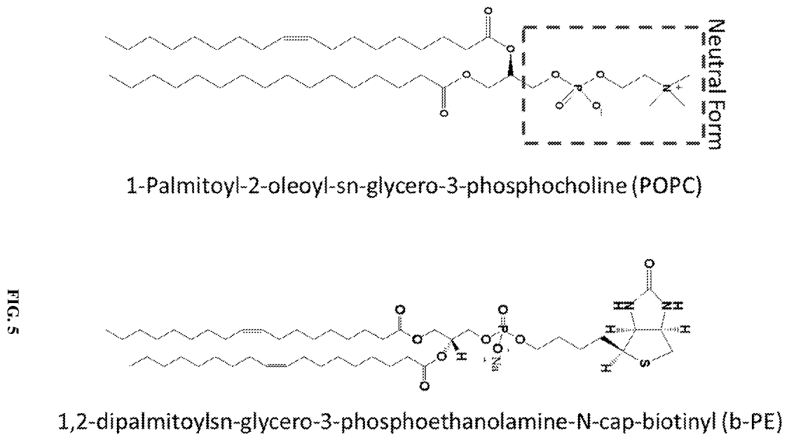

The table in FIG. 4. showed a side-by-side comparison of the results obtained using a 2D chip or the lipid vesicle-coated magnetic beads provided herein. The 2D chip in the first enrichment only achieves a 45-60% recovery rate of target cell and <3000 residual white blood cells. Thus, for the 2D chips, a second enrichment is needed before downstream molecular analysis can be performed. However, more enrichment results in a decreased recovery rate of the target cells. The lipid-coated magnetic beads provided herein demonstrate improved cell capture (>70%) and reduced residual white blood cell number (equal to or less than 300 white blood cells). These data demonstrate that further enrichment of the target cells before downstream molecular analysis is not necessary when the presently provided lipid vesicle-coated magnetic beads are used.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A composition comprising:
(i) a magnetic bead having attached to its exterior surface a plurality of first binding partners;
(ii) a plurality of lipid vesicles that comprise a plurality of the first binding partners on its exterior surface;
(iii) a plurality of second binding partners; and
(iv) a plurality of agents that bind specifically to a target cell, wherein each agent comprises an attached first binding partner;
wherein:
each of the plurality of second binding partners is capable of specifically binding to one or more first binding partners,
a first subset of the plurality of the second binding partners specifically binds to (i) a first binding partner attached to the exterior surface of the magnetic bead and (ii) a first binding partner on the exterior surface of a lipid vesicle;
a second subset of the plurality of the second binding partners specifically binds to (i) a first binding partner on the exterior surface of a lipid vesicle, and (ii) a first binding partner attached to an agent that binds specifically to a target cell.

2. The composition of claim 1, wherein the lipid vesicles are non-fouling lipid vesicles.

3. The composition of claim 1, wherein the non-fouling lipid vesicles comprise a zwitterionic lipid molecule.

4. The composition of claim 1, wherein the non-fouling lipid vesicles comprise polyelectrolyte multilayers (PEMs) or a polymer brush.

5. The composition of claim 4, wherein the PEMs comprise one or more of: poly-L-lysine, poly-L-glutamic acid, and poly-L-aspartic acid.

6. The composition of claim 4, wherein the polymer brush comprises [2-acryloyloxy)ethyl] trimethyl ammonium chloride (TMA) and 2-carboxyethyl acrylate (CAA).

7. The composition of claim 1, wherein the vesicles comprise 1-palmitoyl-2-oleoyl-sn-glycero-3-phosphocholine (POPC) and 1,2-dipalmitoyl-sn-glycero-3-phosphoethanolamine-N-cap-biotinyl (b-PE), and biotin is the first binding partner.

8. The composition of claim 7, wherein the lipid vesicles comprise POPC and b-PE at a ratio of 85:15.

9. The composition of claim 1, wherein the magnetic bead has covalently attached to its exterior surface the plurality of first binding partners.

10. The composition of claim 1, wherein the magnetic bead has non-covalently attached to its exterior surface the plurality of first binding partners.

11. The composition of claim 1, wherein the plurality of agents that bind specifically to a target cell each comprise a covalently attached first binding partner.

12. The composition of claim 1, wherein the plurality of agents that bind specifically to a target cell each comprise a non-covalently attached first binding partner.

13. The composition of claim 1, wherein the first binding partner comprises biotin or a derivative thereof.

14. The composition of claim 1, wherein the second binding partner comprises avidin or a derivative thereof.

15. The composition of claim 1, wherein the plurality of agents that bind specifically to the target cell is an antibody or an antigen-binding fragment thereof.

16. The composition of claim 1, wherein the target cell is a cancer cell, and the plurality of agents that bind specifically to the target cell is an antibody or an antigen-binding fragment thereof that specifically binds to a cancer antigen.

17. The composition of claim 16, wherein the cancer antigen is epithelial cell adhesion molecule (EpCAM).

18. The composition of claim 1, wherein the first binding partner binds to the second binding partner with a disassociation constant (K_{D}) of ≤10⁻⁷ M.

19. The composition of claim 1, wherein the first binding partner binds to the second binding partner with disassociation constant (K_{D}) of ≤10⁻⁹ M.

20. A kit comprising a composition according to claim 1.

21. A method of isolating a target cell from a biological sample comprising:
(a) contacting a biological sample comprising a target cell and non-target cells with a composition of claim 1;
(b) after (a), washing the magnetic bead with a wash buffer under conditions sufficient to allow the association between (i) the first binding partner and the second binding partner to form a complex, and (ii) the agent that binds specifically to the target cell and the complex; and
(c) after (b), applying a magnetic force to the magnetic bead under conditions sufficient to allow the association between (i) the first binding partner and the second binding partner, and (ii) the target cell and the agent that binds specifically to the target cell, thereby isolating the target cell.

22. The method of claim 21, wherein the isolated target cell is viable.

23. The method of claim 21, wherein the target cell is a circulating tumor cell or a circulating tumor stem cell.

24. The method of claim 21, further comprising:
(d) contacting the magnetic bead with an elution buffer under conditions that allow for the disassociation between the target cell and the agent that binds specifically to the target cell, thereby releasing the target cell from the magnetic bead.

25. The method of claim 21, wherein the biological sample comprises blood.

26. The method of claim 21, wherein the biological sample was obtained from a subject that has been diagnosed as having a cancer.

27. The method of claim 21, wherein the biological sample was obtained from a subject that is suspected of having a cancer.

28. The method of claim 21, wherein the wash buffer comprises phosphate buffered saline and bovine serum albumin.

29. The method of claim 28, wherein the wash buffer comprises 1% w/v bovine serum albumin.

30. The method of claim 21, further comprising:
(d) extracting a nucleic acid from the enriched target cell in step (c).

31. The method of claim 30, further comprising:
(e) genotyping the nucleic acid extracted from the enriched target cell in step (d).

32. The method of claim 31, further comprising:
(f) selecting or administering a pharmaceutical treatment to a subject based specifically on the genotype of the nucleic acid extracted from the enriched target cell in step (e).

33. The method of claim 21, wherein the enriched isolated target cell is viable.
